(19) United States
(12) Patent Application Publication
(10) Pub. No.: US 2026/0325320 A1
(43) Pub. Date: Oct. 22, 2026

(54) METHOD FOR CONTROLLING A TRANSTIBIAL PROSTHESIS AND TRANSTIBIAL PROSTHESIS

(71) Applicant: Otto Bock Healthcare Products GmbH, Vienna (AT)

(72) Inventor: Andreas Bohland, Vienna (AT)

(21) Appl. No.: 19/119,281

(22) PCT Filed: Oct. 9, 2023
(86) PCT No.: PCT/EP2023/077905

§ 371 Date: Apr. 8, 2025

(30) Foreign Application Priority Data
Oct. 10, 2022 (DE) 10 2022 126 221.9

(51) Int. Cl. A61F 2/66; A61F 2/68; A61F 2/70; A61F 2/74

(52) U.S. Cl. CPC A61F 2/6607; A61F 2/74; A61F 2002/6818; A61F 2002/704

(57) ABSTRACT

The invention relates to a method for controlling a lower leg prosthesis comprising a foot element, a lower leg element pivotably arranged thereon and an adjustable resistance device for applying a resistance against a swivelling of the foot element relative to the lower leg element, the method comprising the following steps:
identifying the speed of locomotion of the user of the lower leg prosthesis,
determining a maximum point within the stance phase of the step cycle from the identified speed,
increasing the resistance of the resistance device to a maximum when the maximum point is reached,
   wherein the higher the identified speed, the earlier the maximum point is in the stance phase.

The invention relates to a method for controlling a lower leg prosthesis comprising a foot element, a lower leg element pivotably arranged thereon and an adjustable resistance device for applying a resistance against a swivelling of the foot element relative to the lower leg element. The invention also relates to a lower leg prosthesis for carrying out such a method.

Lower leg prostheses controlled by a method described here have been known from the prior art for many years. During the stance phase of a step cycle, i.e. the time in which the foot element is in contact with the ground, a rolling-over of the lower leg element occurs, i.e. a swivelling of the lower leg element relative to the foot element. Plantar flexion begins with heel strike, in which the contact surface with which the foot element makes contact with the ground increases until the foot element is in full contact with the ground, i.e. with the entire sole. The angle between the lower leg element and the foot element becomes larger in the process, such that one refers to a plantar flexion.

This phase is followed by a dorsal flexion, i.e. the movement in the opposite direction. In this phase, the center of gravity of the wearer's body moves over the foot element and the angle between the lower leg element and the foot element becomes smaller. This process is called dorsal flexion and is also referred to as “rolling over”. A healthy human foot also performs these two movements during the stance phase of a step cycle. In a healthy foot, the step cycle ends with an active plantar flexion, i.e. an actively induced increase in the angle between the lower leg and the foot. In this movement phase, the foot pushes itself and thus the person whose foot it is, thus supporting the forward movement.

In the case of a lower leg prosthesis, this active plantar flexion is often no longer possible or is not sufficiently strong, so that the wearer of the prosthesis has to support the forward movement in another way, which often leads to an unnatural gait. Therefore, the foot element usually has at least one spring element, such as a leaf spring made of a fiber-reinforced plastic, such as a carbon fiber-reinforced plastic. Said spring element should be elastically deformed during the final phase of the step cycle and release the energy stored within it as a result at the end of the stance phase, thus accelerating the foot forwards. For this to happen, the resistance that the resistance device sets against the swivelling of the foot element relative to the lower leg element has to be increased. This may be done continuously or in the form of a sudden switch. The point at which the resistance reaches its maximum value is called the maximum point.

In many embodiments, the resistance reached at the maximum point is so great that a further swivelling of the lower leg element relative to the foot element is no longer possible. This is advantageous, but not absolutely essential.

The aim of controlling the lower leg prosthesis is, among other things, to achieve the most natural gait possible at various speeds of locomotion of the user of the lower leg prosthesis. In this case, it must be taken into account that the energy stored in the spring element of the foot element, said energy being necessary for a natural gait, depends on various parameters of movement, such as the speed of the user's locomotion. A range of implementation approaches are known from the prior art. For example, it is known to control the resistance with which the resistance device counters the swivelling as a function of the detected speed of locomotion, wherein resistance is generally reduced the more quickly the wearer of the lower leg prosthesis moves. This is based on the idea that slow movement is similar to standing and as such it is necessary and practical to stabilize the ankle joint. This is especially advantageous in lower leg prostheses whose foot element does not feature a spring element.

Conversely, EP 3 427 701 B1 describes making the maximum point dependent on the identified speed of locomotion. The greater the speed, the later the maximum point is reached in the stance phase. This should lead to a larger range of movement between the lower leg element and the foot element, so that it becomes easier for the wearer of the lower leg prosthesis to move quickly. Furthermore, the movement between the lower leg element and the foot element becomes easier over a longer time period, namely until the maximum point is reached.

Another technical reason for postponing the maximum point with higher speed in the stance phase is that the lower leg prosthesis often contains a hydraulic system and a movement of the lower leg element relative to the foot element causes a hydraulic fluid to move from one chamber of the system into another. The resulting resistance to the movement depends on the viscosity of the hydraulic fluid, which in turn depends on the speed. In the process, viscosity decreases as the speed increases, causing flow resistance to increase. In order to compensate this, the resistance generated by the resistance device is reduced.

However, it is a disadvantage that, particularly when the user of the lower leg prosthesis is moving at high speeds, the amount of energy that is or can be stored in the foot element is often insufficient for natural movement or to sustain the speed. The invention therefore aims to reduce or to completely eliminate this disadvantage.

The invention solves the addressed task by way of a method for controlling a lower leg prosthesis comprising a foot element, a lower leg element pivotably arranged thereon and an adjustable resistance device for applying a resistance against a swivelling of the foot element relative to the lower leg element, the method comprising the following steps:
identifying the speed of locomotion of the user of the lower leg prosthesis,
determining a maximum point within the stance phase of the step cycle from the identified speed,
increasing the resistance of the resistance device to a maximum when the maximum point is reached,
wherein the higher the identified speed, the earlier the maximum point is in the stance phase.

The maximum point is therefore a function of the identified speed at which the user of the lower leg prosthesis moves. As the speed increases, the maximum point is displaced forwards within the stance phase, i.e. at earlier points in time. This means that the value of the maximum point within the stance phase is a monotonically decreasing function with increasing speed. The value of the maximum point within the stance phase is a strictly monotonically decreasing function with increasing speed, at least in sections. Preferably, it is a strictly monotonically decreasing function across the entire speed range. In this context, a decreasing function means that as the speed increases, the maximum point is shifted to earlier points in time within the stance phase.

Within the scope of the present invention, increasing the resistance to the maximum value when the maximum point is reached means that the resistance is increased at this point if the resistance assumes a smaller value. If the resistance already assumes the maximum value at this point, an amendment of the resistance is not necessary and is not carried out.

However, the resistance of the resistance device usually has to be increased to reach the maximum value. To this end, the adjustable resistance device generally has to be adjusted in order to adjust the resistance. This takes a certain amount of time, so that in this case even the resistance does not reach its maximum value at the maximum point. Consequently, the higher the speed at which the user of the lower leg prosthesis moves, the earlier the swivelling of the lower leg element relative to the foot element is made more difficult by increasing the resistance of the resistance device to the maximum value. As a result, the higher the speed, the more energy is stored in an elastic element of the foot element, such as a carbon spring; released again at a later point; converted into motion energy, and made available to the foot. Surprisingly, the movement sequence is better harmonized with the natural movement.

The lower leg prosthesis has an electrical control unit that is configured to carry out the steps in the method.

Preferably, the maximum point corresponds to a switching angle between the foot element and the lower leg element and/or an absolute angle of the lower leg element in the dorsal flexion phase of the stance phase of the step cycle, wherein the switching angle is greater the greater the identified speed. In the dorsal flexion phase, the angle between the lower leg element and the foot element continuously decreases. The greater the switching angle, the earlier in the stance phase the resistance reaches the maximum value. In this case, the “time” at which the maximum point is reached is therefore not measured in seconds or another unit of time, but corresponds to an angle reached during the stance phase. For example, this angle is an angle between the lower leg element and the foot element. As soon as this angle, which is also referred to as the ankle angle, reaches a predetermined value, the maximum point is deemed reached and the resistance increased to the maximum value. If the ankle angle is used as a criterion for reaching the maximum point, it is irrelevant how many seconds have passed before this ankle angle is reached. A larger ankle angle is considered an earlier maximum point.

Alternatively or additionally, an absolute angle of the lower leg element is used as a switching angle. Said angle can be determined via absolute angle sensors. Preferably, such a sensor has an inertial measurement unit (IMU). Alternatively or additionally, the sensor has a combination of accelerometers and/or gyroscopes from which the orientation of the lower leg element in space and thus the absolute angle is determined. Alternatively or additionally, the lower leg prosthesis has at least one sensor that is configured to determine the absolute angle of the foot element and a relative angle between foot element and lower leg element. The combination of these two angles is preferably used to determine the absolute angle of the lower leg element.

The value identified in this way is compared with a fixed value for the switching angle so that it can be determined within the electronic control unit by means an algorithm whether the switching angle has been reached. An absolute angle provides information on the absolute position of an object, i.e. the lower leg element in the present case, in space. It is measured against a fixed base direction. For example, if an object is vertically oriented, i.e. its longitudinal direction follows the direction given by gravity, it has an absolute angle of 0° if the direction given by gravity, i.e. the vertical, is the base direction. Conversely, if the base direction is the horizontal that is perpendicular to the vertical, the absolute angle is also 90° when the object has a vertical orientation.

In one preferred embodiment, the speed of forward movement is determined by identifying a step length or step duration, wherein the identified step length and/or identified step duration is allocated to a speed. The greater the identified step length and/or the shorter the step duration, the greater the speed allocated. Instead of the step duration, the so-called cadence, i.e. the reciprocal value of the step duration, which is specified in Hertz, is often used. This also is considered as using the step duration in the context of the present invention.

In one preferred embodiment, the absolute angle of the lower leg element at the point of heel strike is used to determined the step length, wherein a step length and thus a speed is allocated to the absolute angle measured.

Preferably, an angular range is determined which the absolute angle of the lower leg element covers during a swing phase of a step cycle, wherein the step length is determined from this determined angular range. For example, the angular range can be identified by determining the absolute angle of the lower leg element at the point of heel strike and the point of so-called “toe-off”, i.e. the point at which the foot element loses contact with the ground The difference can be formed from these two measurements and the angular range thus determined.

Preferably, an angular speed of the absolute angle of the lower leg element is measured in the swing phase of the step cycle. Here, the speed of locomotion is determined from said angular speed or a speed is allocated to the angular speed. The angular speed refers to the first deviation of the absolute angle of the lower leg element with respect to time. It refers therefore to the change in the absolute angle in a predetermined time unit. The greater this deviation, the greater also the speed of locomotion that is determined from or allocated to this change. Alternatively or additionally, the speed of locomotion is measured by means of at least one gyroscope, preferably multiple gyroscopes.

Preferably, the resistance of the resistance device is not decreased again until the stance phase of the following step cycle. Particularly preferably, this decrease occurs with the heel strike in the following step cycle. As a result, the resistance of the resistance device remains at the maximum value, even during the swing phase between the two consecutive stance phases, and a swivelling of the lower leg element relative to the foot element is rendered more difficult or completely impossible. Consequently, the lower peg prosthesis remains in the swing phase in the position assumed at the maximum point. This means that the lower leg element and foot element remain orientated almost or completely unchanged in relation to each other. This is especially advantageous if the maximum point is reached when the angle between the lower leg element and the foot element is smaller than 90°. In this case, the direction of the lower leg element corresponds to the longitudinal direction of the prosthetic component used as a lower leg element. The direction of the foot element corresponds to the direction of the tread surface of the foot element, i.e. the surface that can be brought into contact with the ground. The further dorsally reflected the ankle angle is, i.e. the further the forefoot is lifted, the more stumbling is prevented during the swing phase by maintaining the maximum resistance value.

In one preferred embodiment, upon reaching the maximum point, the resistance is maintained at a value that is so high that a further swivelling of the foot element relative to the lower leg element is no longer possible if the speed determined exceeds a predetermined limit value. It is particularly preferable if the resistance does not decrease again until the speed determined falls back below the predetermined limit value. As a result, when the wearer of the lower leg prosthesis walks quickly, for example, the resistance is permanently so high that the foot element cannot be moved relative to the lower leg element. This means that a stance phase does not begin with a heel strike, but contact between the forefoot of the foot element and the ground already occurs at this early point in the stance phase. This corresponds to the natural movement of a healthy person who only touches the ground with the forefoot area, i.e. mainly with their toes, when walking quickly.

The invention also solves the task addressed by way of a lower leg prosthesis with a foot element, a lower leg element pivotably arranged thereon and an adjustable resistance device for applying a resistance against a swivelling of the foot element relative to the lower leg element, wherein the lower leg prosthesis comprises at least one sensor for determining measurement data and an electrical control unit that is configured to determine the speed of locomotion from the measurement data and to conduct a method according to one of the preceding claims. The lower leg prosthesis has an electrical control unit, preferably in the form of an electronic data processing device, which is configured to carry out one of the methods given above.

The resistance device preferably has a hydraulic system. Said system contains a hydraulic fluid that moves within the hydraulic system during movement of the lower leg element relative to the foot element. The hydraulic system then counters the movement of the fluid with a flow resistance. This flow resistance can be adjusted via an actuator. Such an actuator is designed, for example, as a throttle valve whose flow area is adjustable.

Alternatively or additionally, the resistance device comprises a magnethorheological device and/or an electric motor.

In the following, an embodiment of the present invention will be explained in more detail with the aid of the accompanying illustrations. They show:

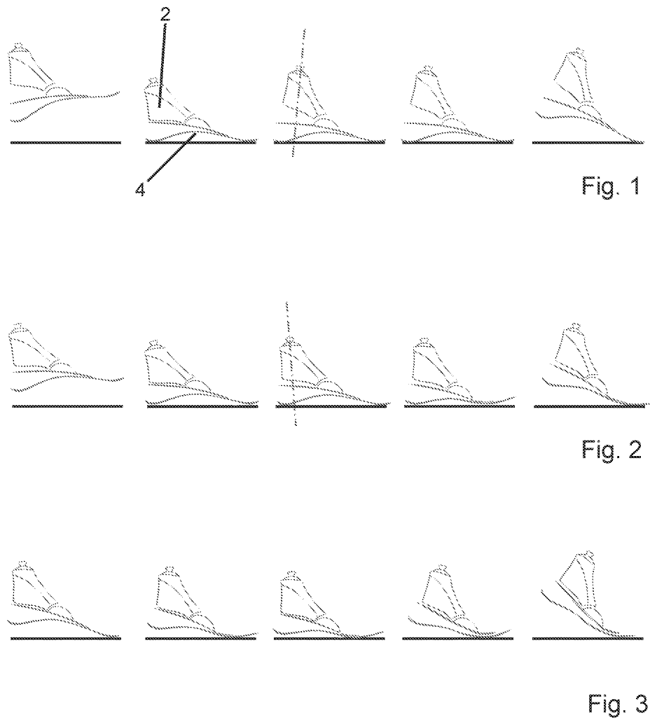

FIGS. 1 to 3—v arious phases in a step cycle at different speeds of locomotion.

FIGS. 1 to 3 each depict five representations of different phases in a step cycle. Going from left to right, in each case they show the end of the swing phase (“Terminal Swing”), the loading response (“Loading Response”), the middle stance phase (“MidStance”), the end of the stance phase (“Terminal Stance”) and the start of the swing phase (“Initial Swing”). FIG. 1 shows the progression during slow walking, FIG. 2 during fast walking and FIG. 3 during running.

Each figure shows a lower leg prosthesis with a lower leg element 2 and a foot element 4, which are arranged so that they can swivel against each other about a swivel axis.

The lower leg prosthesis has a resistance device, not depicted here, by means of which a resistance can set against the swivelling of the two elements 2,4 relative to each other. This resistance can be adjusted via the resistance device.

FIG. 1 depicts the progression during slow walking. At the end of the swing phase (leftmost representation), the resistance device is set so that there is little resistance to the swivelling of the foot element 4 relative to the lower leg element 2. In one preferred embodiment, the resistance device only applies a low resistance in this situation. During the loading response (second representation from the left), the ankle has a large range of movement. In the representation shown, the lower leg element is widely plantar-flexed, i.e. swivelled anti-clockwise relative to the foot element 4 in the representation shown. It is clear that the lower leg element 2 is shown to be very close to an upper spring of the foot element 4. A deformation of the spring elements on the heel of the foot element 4 that also occurs is not shown in any of the representations. Later on in the stance phase (the middle representation in FIG. 1), the lower leg element 2 is dorsal-flexed relative to the foot element 4. It is not until a relatively late point in this movement that the maximum point is reached, from which the resistance caused by the resistance device is increased to such an extent that, in the embodiment example shown, a further swivelling of the lower leg element 2 relative to the foot element 4 is no longer possible. During slow walking (FIG. 1), this does not happen until towards the end of the stance phase (second representation from the right). It is clear that an angle between the lower leg element 2 and the foot element 2 continues to decrease up until this point, which corresponds to an increasing distance between the lower leg element 2 and the upper spring of the foot element 4 in the heel area. Since the maximum point is reached very late or, in one preferred embodiment, the lower leg element 2 can be swivelled relative to the foot element 4 up to the mechanical end stop, the movement after this point in time is only slight, so that the springs of the foot element 4 can only be slightly tensioned. As a result, only a little energy can be released at the start of the swing phase (rightmost representation). During the swing phase, no more energy is released and a movement of the lower leg element 2 relative to the foot element 4 does not occur or not to any significant extent. The position of the lower leg element 2 relative to the foot element 4 in this phase therefore corresponds to the position at which the start of the next stance phase (leftmost representation) is initiated.

FIG. 2 shows the same representations for when the wearer of the prosthesis is walking quickly. It is clear that the start of the stance phase (leftmost representation) begins with a considerably flatter foot strike. The foot is more starkly plantar-flexed than in FIG. 1 during slow walking. This situation occurs when the preceding step was also already executed at the higher speed. Even during fast walking, a movement of the lower leg element 2 relative to the foot element 4 initially possible. In simple terms, in the loading response (second representation from the left), the position of the lower leg element 2 relative to the foot element 4 does not differ to the corresponding position during slow walking. However, during rolling-over in the middle of the stance phase (middle representation), the maximum point is achieved significantly earlier than during slow walking. As a result, the resistance device blocks a further swivelling of the lower leg element 2 relative to the foot element 4 from this point in time by way of the resistance it applies. At the very least, however, the applied resistance makes this further swivelling more difficult. It can therefore be seen that the distance between the lower leg element 2 and the foot element 4 in the middle of the stance phase is significantly smaller during fast walking than during slow walking. This means that the springs of the foot element 4 are deformed during further rolling-over. This can be clearly seen at the end of the stance phase (second representation from the right).

Consequently, more potential energy is stored in the springs, which is released at the start of the swing phase (representation on the far right). As a result, the foot element 4 is held in a much more plantar-flexed position relative to the lower leg element 2 than when walking slowly. Even when walking fast, there is no further movement of the foot element 2 relative to the lower leg element 4 during the swing phase, so that here too the position at the start of the swing phase (rightmost representation) corresponds to the position at the end of the swing phase (leftmost representation).

FIG. 3 shows the situation when running. In the embodiment shown, the speed is so great that the maximum point has been reached very early. If the earliest possible maximum point is reached over several steps, the resistance device is controlled in such a way that the resistance it generates remains constantly high. In the embodiment example shown, this means that a swivelling of the lower leg element 2 relative to the foot element 4 is no longer possible and the orientation and position of the two elements relative to each other does not change. All rolling movements are therefore made possible by the deformation of the springs. This means, for example, that the start of the stance phase, i.e. the end of the swing phase (leftmost representation), does not begin with a heel strike but with a toe strike. There is no contact between the heel and the ground during the entire stance phase (second representation from the left to the second representation from the right). The entire energy from the forefoot load is stored in the deformed springs at the end of the stance phase (second representation from the right) and is released at the beginning of the swing phase (rightmost representation).

### Reference list

Lower leg element 2
Foot element 4

1. A method for controlling a lower leg prosthesis comprising a foot element (4), a lower leg element (2) pivotably arranged thereon and an adjustable resistance device for applying a resistance against a swivelling of the foot element (4) relative to the lower leg element (2), the method comprising the following steps:
identifying the speed of locomotion of the user of the lower leg prosthesis,
determining a maximum point within the stance phase of the step cycle from the identified speed,
increasing the resistance of the resistance device to a maximum when the maximum point is reached,
wherein the higher the identified speed, the earlier the maximum point is in the stance phase.

2. The method according to claim 1, characterized in that the maximum point corresponds to a switching angle between the foot element (4) and the lower leg element (2) and/or an absolute angle of the lower leg element (2) in the dorsal flexion phase of the stance phase of the step cycle, wherein the switching angle is greater the greater the identified speed.

3. The method according to claim 1, characterized in that the speed of locomotion is determined by identifying a step length and/or step duration, and the identified step length and/or identified step duration is allocated to a speed, wherein the greater the identified step length and/or the shorter the step duration, the greater the speed allocated.

4. The method according to claim 3, characterized in that an absolute angle of the lower leg element (2) is measured at the point of heel strike and the step length is determined from said absolute angle.

5. The method according to claim 3, characterized in that an angular range is determined which the absolute angle of the lower leg element (2) covers during a swing phase of a step cycle, and that the step length is determined from this angular range.

6. The method according to claim 1, characterized in that an angular speed of the absolute angle of the lower leg element (2) is measured in a swing phase of a step cycle and the speed of locomotion is determined from said angular speed.

7. The method according to claim 1, characterized in that the resistance of the resistance device is only decreased again in the stance phase of the following step cycle.

8. The method according to claim 1, characterized in that, upon reaching the maximum point, the resistance is kept so high that a further swivelling of the foot element (4) relative to the lower leg element (2) is no longer possible if the speed determined exceeds a predetermined limit value.

9. The method according to claim 8, characterized in that the resistance does not decrease again until the speed determined falls back below the predetermined limit value.

10. A lower leg prosthesis with a foot element (4), a lower leg element (2) pivotably arranged thereon and an adjustable resistance device for applying a resistance against a swivelling of the foot element (4) relative to the lower leg element (2), wherein the lower leg prosthesis comprises at least one sensor for determining measurement data and an electrical control unit that is configured to determine the speed of locomotion from the measurement data and to conduct a method according to claim 1.

11. The lower leg prosthesis according to claim 10, characterized in that the resistance device has a hydraulic system.

12. The lower leg prosthesis according to claim 10 characterized in that the resistance device has a magnetorheological device.

13. The lower leg prosthesis according to claim 10 to characterized in that the resistance device has an electric motor.
